(19) United States
(12) Patent Application Publication
(10) Pub. No.: US 2022/0087733 A1
(43) Pub. Date: Mar. 24, 2022

(54) ELONGATED MEDICAL NEEDLE

(71) Applicant: Baylis Medical Company Inc., Montreal (CA)

(72) Inventor: Suleman Azam, Waterloo (CA)

(21) Appl. No.: 17/460,854

(22) Filed: Aug. 30, 2021

Related U.S. Application Data
(60) Provisional application No. 63/081,942, filed on Sep. 23, 2020.

(51) Int. Cl. A61B 18/14

(52) U.S. Cl. CPC A61B 18/1477; A61B 2018/00357; A61B 18/1487

(57) ABSTRACT

An elongated medical needle defines an open lumen presenting a distal face configured to be maneuvered toward, and positionable proximate to, the biological feature of the patient. A lid assembly is configured to selectively cover, at least in part, the distal face of the open lumen. The lid assembly is also configured to selectively emit energy toward the biological feature.

### TECHNICAL FIELD

This document relates to the technical field of (and is not limited to) an elongated medical needle (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing (known) radiofrequency medical needles. After much study of, and experimentation with, the existing (known) radiofrequency medical needles, an understanding (at least in part) of the problem and its solution have been identified (at least in part) and are articulated (at least in part) as follows:

Referring to the embodiments as depicted in FIG. 1 and FIG. 2 (PRIOR ART), there are depicted perspective views of a known mechanical needle 800 having an open lumen 802. The open lumen 802 is configured to facilitate movement of an anchoring guidewire (not depicted) to be tracked along the shaft of the known mechanical needle 800, and to anchor the puncture location once (or when) the septum is initially crossed, etc. The open lumen 802 also allows a contrast material to be directly delivered onto the septum, thereby improving user visibility of a target site (viewable from under fluoroscopy imaging). Further, this characteristic allows the user to perform pressure measurements to confirm the location of the needle within the heart. Physicians use the known mechanical needle 800 that requires application of a mechanical force, from the user, to puncture the interatrial septum, etc.

Referring to FIG. 1, the open lumen 802, in use, tents the fossa ovalis, and partially vaporizes the tissue. Referring to FIG. 2, the open lumen 802 introduces a particulate of tissue 803 (also called a tissue core) that may lead, unfortunately, to an embolism (after the open lumen 802 partially vaporizes the tissue).

An issue with implementing the open lumen 802 is the unwanted prospect of the formation of a tissue core during the formation of the puncture hole. The open lumen 802 may be configured to be electrically-active (by emitting energy) to vaporize the tissue contacting the ring-shaped active surface of the open lumen 802. The tissue positioned inside the open lumen 802 (the ring) is not vaporized and, instead, may become unfortunately separated from the tissue wall, and thereby become a free-floating particulate of tissue 803, which might introduce the risk of causing stroke or pulmonary embolism in the patient. The known mechanical needle 800 (also called an open lumen radiofrequency needle) may need a method to combat this unwanted risk.

Referring to the embodiment as depicted in FIG. 3 (PRIOR ART), there is depicted a perspective view of a known radiofrequency needle 810 (puncture device), such as the NRG (TRADEMARK) RF Transseptal Needle manufactured by the BAYLIS (TRADEMARK) Company (headquartered in Canada), as an alternative to the embodiments depicted in FIG. 1 and FIG. 2. Physicians have adopted the known radiofrequency needle 810 for puncturing the interatrial septum (a biological feature) to achieve transseptal access into the left side of the heart of the patient. The known radiofrequency needle 810 is configured to deliver (emit) radiofrequency energy via an active electrode positioned at a distal tip (of the radiofrequency needle) to vaporize a target tissue (and thereby form a puncture hole through a biological feature). However, it is still common for physicians to use the known mechanical needle 800 that requires application of a mechanical force, from the user, to puncture the interatrial septum, etc. Advantages of using the known radiofrequency needle 810 may include: improved precision for the location of forming a puncture through the biological feature, reduced risk of tissue damage and/or tears, and/or reduced application of a mechanical force required from the user (for the formation of the puncture hole). It will be appreciated that there are factors that might prevent physicians from adopting the known radiofrequency needle 810 (of FIG. 3) over the known mechanical needle 800 (of FIG. 1 and FIG. 2).

To mitigate, at least in part, at least one problem associated with the existing technology, there is provided (in accordance with a major aspect) an apparatus. The apparatus is for use with a biological feature of a patient. The apparatus includes and is not limited to (comprises) an elongated medical needle defining an open lumen presenting a distal face configured to be maneuvered toward, and positionable proximate to, the biological feature of the patient. A lid assembly is configured to selectively cover (occlude), at least in part, the distal face of the open lumen. The lid assembly is also configured to puncture through the biological feature. The lid assembly is also configured to selectively uncover, at least in part, the distal face of the open lumen after the lid assembly has punctured through the biological feature.

To mitigate, at least in part, at least one problem associated with the existing technology, there is provided (in accordance with a major aspect) an apparatus. The apparatus is for use with a biological feature of a patient. The apparatus includes and is not limited to (comprises) an elongated medical needle. The elongated medical needle defines an open lumen presenting a distal face. The distal face is configured to be maneuvered toward, and positionable proximate to, the biological feature of the patient. A lid assembly is configured to selectively cover (occlude), at least in part, the distal face of the open lumen. The lid assembly is also configured to selectively emit energy toward the biological feature (in response to receiving energy from a known energy source); this is done, preferably, after the distal face has been maneuvered toward, and positioned proximate to, the biological feature and after the lid assembly, in use, selectively covers, at least in part, the distal face of the open lumen; this is done, preferably, in such a way that the lid assembly, in use, vaporizes a portion of the biological feature.

To mitigate, at least in part, at least one problem associated with the existing technology, there is provided (in accordance with a major aspect) a method. The method is for emitting energy (such as radiofrequency energy) toward a biological feature of a patient. The method includes and is not limited to (comprises) maneuvering and positioning a distal face of an open lumen of an elongated medical needle toward and proximate to the biological feature of the patient. The method also includes selectively covering, at least in part, the distal face of the open lumen with a lid assembly. The method also includes selectively emitting energy toward the biological feature from the lid assembly (this is done, preferably, after the distal face has been maneuvered toward, and positioned proximate to, the biological feature and after the lid assembly, in use, selectively covers, at least in part, the distal face of the open lumen); this is done, preferably, in such a way that the lid assembly, in use, vaporizes a portion of the biological feature.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 and FIG. 2 depict perspective views known mechanical needle; and

FIG. 3 depict perspective view of a known radiofrequency needle; and

FIG. 4, FIG. 5 and FIG. 6 depict a perspective view (FIG. 4) and top views (FIG. 5 and FIG. 6) of embodiments (implementations) of an elongated medical needle; and

FIG. 7, FIG. 8 and FIG. 9 depict perspective views of embodiments (implementations) of the elongated medical needle of FIG. 4; and

FIG. 10, FIG. 11 and FIG. 12 depict schematic views of embodiments (implementations) of the elongated medical needle of FIG. 4; and

FIG. 13 and FIG. 14 depict perspective views of embodiments (implementations) of the elongated medical needle of FIG. 4; and

FIG. 15, FIG. 16, FIG. 17 and FIG. 18 depict perspective views of embodiments (implementations) of the elongated medical needle of FIG. 4.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

elongated medical needle 102
open lumen 104
side wall 105
distal face 106
lid assembly 108
wire structure 110
storage compartment 112
lumens (114A, 114B)
mechanical needle 800
open lumen 802
particulate of tissue 803
radiofrequency needle 810
biological feature 900
patient 902

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

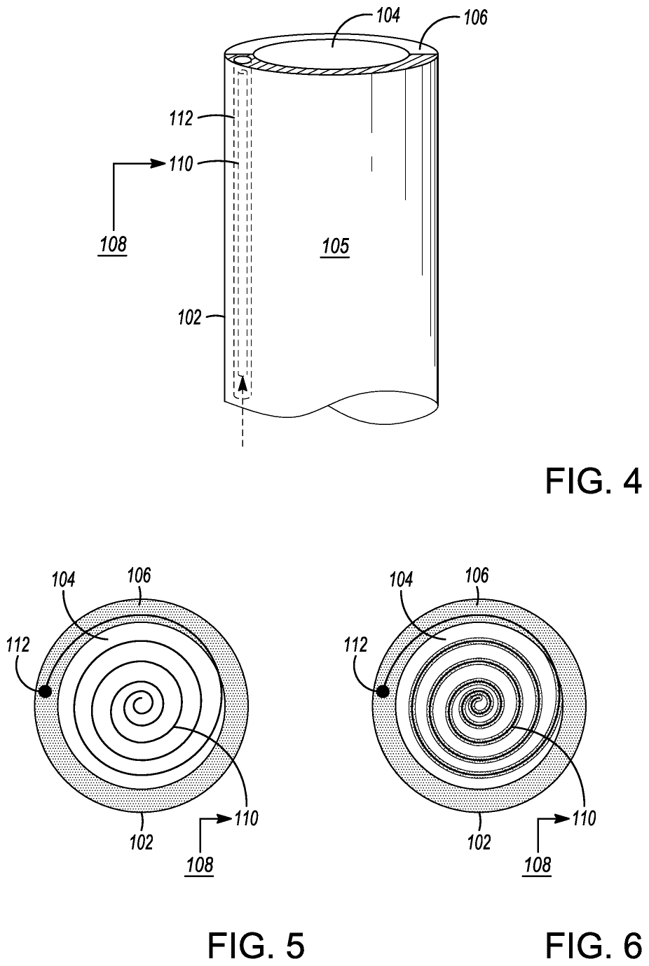

FIG. 4, FIG. 5 and FIG. 6 depict a perspective view (FIG. 4) and top views (FIG. 5 and FIG. 6) of embodiments (implementations) of an elongated medical needle 102.

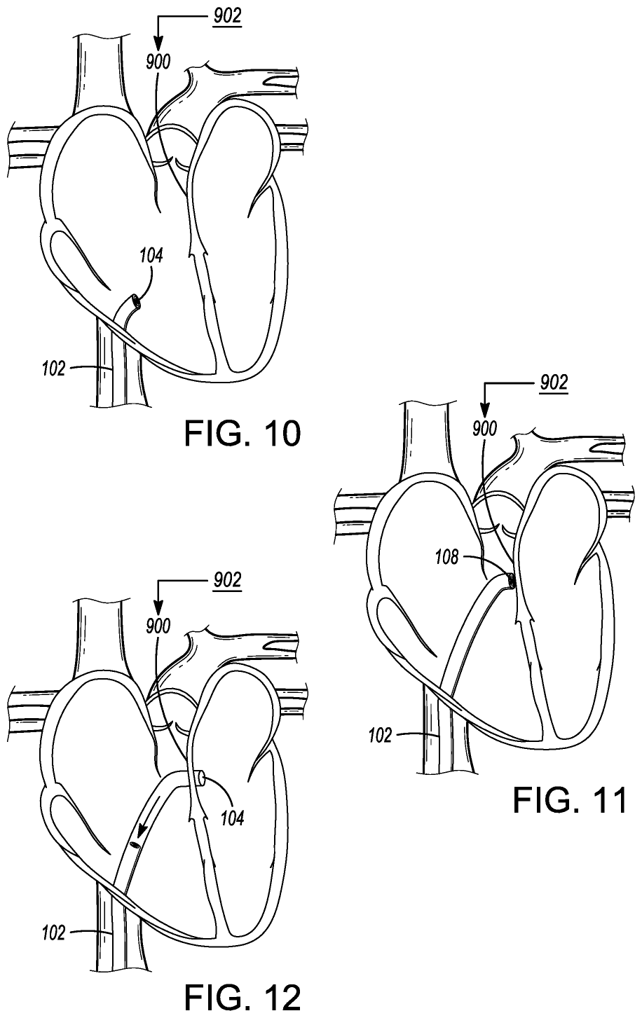

Referring to the embodiment (implementation) as depicted in FIG. 4, the apparatus is for use with a biological feature 900 of a patient 902 (as depicted in FIG. 10 to FIG. 14). The apparatus includes and is not limited to (comprises) an elongated medical needle 102. The elongated medical needle 102 defines an open lumen 104 presenting a distal face 106. The distal face 106 is configured to be maneuvered toward, and positionable proximate to, the biological feature 900 of the patient 902 (as depicted in FIG. 10 to FIG. 14). A lid assembly 108 is configured to selectively cover (occlude), at least in part, the distal face 106 of the open lumen 104 (as depicted in FIG. 5 and FIG. 6). The lid assembly 108 is also configured to selectively emit energy toward the biological feature 900 (in response to receiving energy from a known energy source, as depicted in FIG. 11); this is done, preferably, after the distal face 106 has been maneuvered toward, and positioned proximate to, the biological feature 900 and after the lid assembly 108, in use, selectively covers, at least in part, the distal face 106 of the open lumen 104 (as depicted in FIG. 11); this is done, preferably, in such a way that the lid assembly 108, in use, vaporizes a portion of the biological feature 900 (as depicted in FIG. 11). In accordance with a preferred embodiment, the lid assembly 108 includes a wire structure 110. The wire structure 110 is, preferably, configured to be stored in the elongated medical needle 102 (preferably, stored in a storage compartment 112 (such as, a channel, etc.) formed along a side wall of the elongated medical needle 102). The wire structure 110 is configured to be moved (deployed from) the storage compartment 112 to an exterior distal zone positioned in front of the exit port of the elongated medical needle 102 (as depicted in FIG. 5 and/or FIG. 6). As depicted in FIG. 4, the wire structure 110 forms, and is stored, as a straight wire; as depicted in FIG. 5 or FIG. 6, the wire structure 110 is deployed and forms (at least in part) a coil structure positioned at the portal of the elongated medical needle 102 (that is, at the distal face 106. The definition of the shaped wire structure 110 may include a pre-set arrangement or a pre-set wire, etc.; the shaped wire structure 110 (or coil shaped wire) may be pre-set even if the shaped wire structure 110 is completely straight due to the constrained space when the shaped wire structure 110 is placed in storage (such as in the lumen, etc.). Once the shaped wire structure 110 is deployed, the shaped wire structure 110 is configured to (naturally) form a coil or other equivalent shape.

Referring to the embodiment (implementation) as depicted in FIG. 4, an advantage of the elongated medical needle 102 is the reduction (preferably, the prevention), at least in part, of the formation of a tissue core (in sharp contrast to the embodiment as depicted in FIG. 2 of the prior art) during, or as a result of, puncturing the biological feature 900 (tissue). The elongated medical needle 102 reduces, at least in part, the possibility of unwanted formation of tissue coring (of the biological feature 900, as depicted in FIG. 12) and subsequent detachment of a free-floating tissue portion (tissue core) from the biological feature 900 (in sharp contrast to the embodiment as depicted in FIG. 2).

Referring to the embodiment (implementation) as depicted in FIG. 4, the elongated medical needle 102 defines the open lumen 104 presenting the distal face 106 configured to be maneuvered toward, and positionable proximate to, the biological feature 900 of the patient 902. The lid assembly 108 is configured to selectively cover (occlude), at least in part, the distal face 106 of the open lumen 104 (as depicted in FIG. 5 or FIG. 6). The lid assembly 108 is also configured to puncture through the biological feature 900. The lid assembly 108 is also further configured to selectively uncover, at least in part, the distal face 106 of the open lumen 104 after the lid assembly 108 has punctured through the biological feature 900 (as depicted in FIG. 4).

Referring to the embodiment (implementation) as depicted in FIG. 4, the lid assembly 108 includes a proximal end (not depicted) that is user accessible, for permitting connection of the lid assembly 108 to the energy source, etc. For instance, the lid assembly 108, once deployed, as depicted in FIG. 5 or FIG. 6, is activated (selectively activated) to selectively emit energy (radiofrequency energy) toward the biological feature 900 (in response to receiving radiofrequency energy from a known radiofrequency energy source that is electrically connected to the proximal portion of the lid assembly 108).

Referring to the embodiment (implementation) as depicted in FIG. 4, the elongated medical needle 102 (and components thereof) includes biocompatible materials having properties suitable for sufficient performance (such as, dielectric strength, thermal characteristics, thermal insulation, corrosion, water resistance, heat resistance, etc.), for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 4, there is depicted a circumferential profile of the distal face 106 (also called a distal end) of the open lumen 104 of the elongated medical needle 102. The elongated medical needle 102 is configured to house the lid assembly 108 (in a storage position). The lid assembly 108 is configured to be movable between the storage position (as depicted in FIG. 4) and the deployed position (as depicted in FIG. 5 or FIG. 6). The lid assembly 108 is configured to selectively advanced, or selectively retracted, along a length of the elongated medical needle 102 (as needed). The lid assembly 108 is configured to selectively emit energy (radiofrequency energy) (once the lid assembly 108 is operatively coupled to a suitable energy source). The distal face 106 is configured to selectively emit energy (the distal face 106 includes an electrically conductive material). The elongated medical needle 102 is configured to house the lid assembly 108 (as depicted in FIG. 4). The lid assembly 108 is operatively mounted (positioned) at the distal face 106 of the open lumen 104 of the elongated medical needle 102. The elongated medical needle 102 is configured to house the lid assembly 108.

Referring to the embodiments (implementations) as depicted in FIG. 5 and FIG. 6, the elongated medical needle 102 and/or the lid assembly 108 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus applied to the shape-memory material. This is known as the shape memory effect (SME). Superelasticity (in alloys) may be observed once the shape-memory material is deformed under the presence (an application) of a stimulus force.

Referring to the embodiments (implementations) as depicted in FIG. 5 and FIG. 6, the distal face 106 is configured (preferably) to be blunted and/or have a tapered profile. A blunted instance of the distal face 106 mitigates, at least in part, the potential of unwanted scraping (skiving) of the interior of a plastic accessory device (known and not depicted) along which the elongated medical needle 102 may be advanced through. A blunted instance of the distal face 106 may improve, at least in part, the efficacy for the crossing of the blunted instance of the distal face 106 through a biological feature (such as, the septum) as the hole puncture size may be relatively smaller than the overall diameter of (such as the proximal section of) the elongated medical needle 102. A tapered profile of the distal face 106 may allow the puncture hole to gradually expand when the elongated medical needle 102 crosses the biological feature (such as the interatrial septum of the heart). In accordance with an option, the distal face 106 may include an electrically conductive material (for assisting the lid assembly 108 in puncture formation by emission of energy from the distal face 106, if so desired). The elongated medical needle 102 includes, preferably, SAE (Society of Automotive Engineering) Type 304 Stainless Steel and/or any equivalent thereof. SAE Type 304 stainless steel contains both chromium (from between about 15% to about 20%) and nickel (from between about 2% to about 10.5%) metals as the main non-iron constituents. The guidewire may include (if desired) a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus that is applied to the shape-memory material. This is known as the shape memory effect (SME). Superelasticity (in alloys) may be observed once the shape-memory material is deformed under the presence (an application) of a stimulus force.

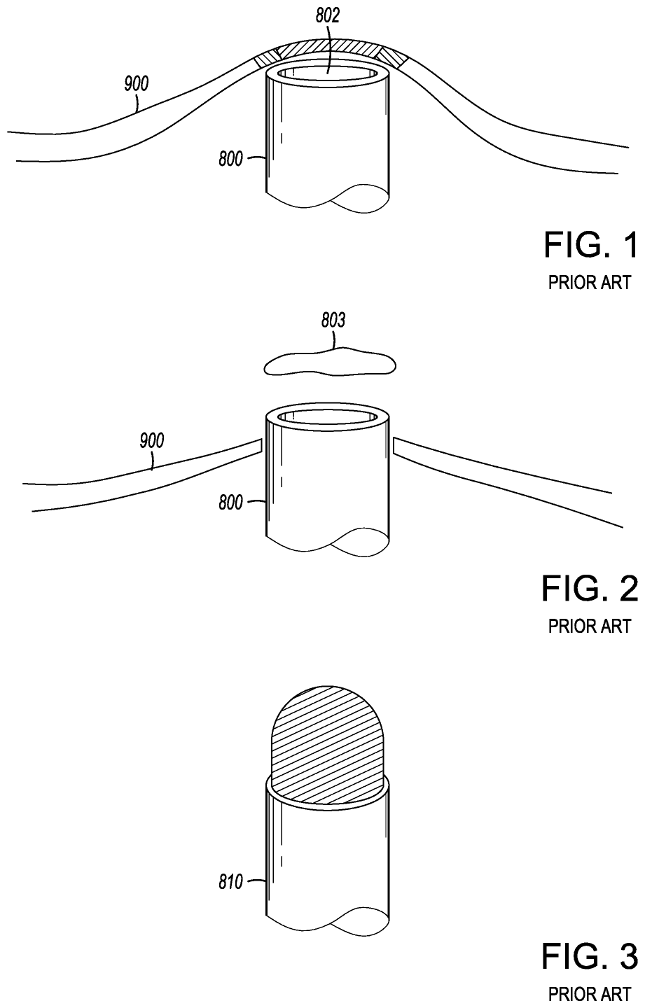

Referring to the embodiments (implementations) as depicted in FIG. 5 and FIG. 6, the lid assembly 108 includes (preferably) a shaped wire structure 110 (or a wire structure) having a memory shape metal, etc., and any equivalent thereof. The shaped wire structure 110 may include a stand-alone wire with a coiled distal end section. The coiled end (of the coiled distal end section) is configured to allow the shaped wire structure 110 to be straightened when advanced through an accessory device (known and not depicted). The shaped wire structure 110 is configured to selectively occlude the distal face 106 of the open lumen 104. The lid assembly 108 has a shape and/or an arrangement (configured) to prevent, at least in part, tissue coring (as depicted in FIG. 1 and FIG. 2) when puncturing the biological feature.

Referring to the embodiments (implementations) as depicted in FIG. 5 and FIG. 6, the lid assembly 108 is configured to be selectively moved away from the distal face 106 in such a way that the distal face 106 may be exposed (opened) to allow the injection of a contrast fluid and/or facilitate a pressure reading, etc., and to facilitate other procedures and/or devices. In accordance with an option, the lid assembly 108 is configured to selectively emit energy (such as radiofrequency energy). In accordance with another option, the lid assembly 108 presents a mechanical puncturing portion (as an option for the emission of energy) provided that the mechanical puncturing portion avoids (at least in part) the formation of tissue coring (as depicted in FIG. 1 and FIG. 2).

Referring to the embodiments (implementations) as depicted in FIG. 5 and FIG. 6, the lid assembly 108 may include Nitinol (shape set) or an SAE 304 stainless steel (cold worked) material. The shaped wire structure 110 may include a shape set Nitinol or cold worked SAE 304 stainless steel to provide the required stiffness and strength to withstand selective retraction and advancement of the shaped wire structure 110 (or the lid assembly 108) through an accessory device (known and not depicted).

Referring to the embodiments (implementations) as depicted in FIG. 5 and FIG. 6, the medical needle 102 may include a hypotube. The medical needle 102 may define or provide a groove extending along a length of the medical needle 102. The lid assembly 108 is configured to be received (at least in part) within (and is movable along) the groove, thereby embedding or storing the lid assembly 108 within the medical needle 102 (ready for deployment therefrom, as depicted in FIG. 5 or FIG. 6). Alternatively, the lid assembly 108 may be housed against the outside surface of (the shaft of) the elongated medical needle 102. The arrangement for housing the lid assembly 108 may allow the user to selectively advance and/or retract the lid assembly 108 from the distal end of the elongated medical needle 102 (as needed or required).

Referring to the embodiments (implementations) as depicted in FIG. 5 and FIG. 6, a length of the elongated medical needle 102 may be covered with a heat shrink material (polytetrafluoroethylene or PTFE heat shrink), with an option for leaving the distal face 106 (the distal tip) unexposed, if so desired. It will be appreciated that PTFE heat shrink offers improved lubricity to ease selective advancement and/or retraction of the elongated medical needle 102 from an accessory device (known and not depicted) and/or the patient's vasculature. The proximal end of the elongated medical needle 102 may include a molded plastic handle (known and not depicted). This handle is configured to allow the user to manipulate the elongated medical needle 102 from the proximal end of the elongated medical needle 102. The lid assembly 108 includes a connection configured to be selectively connected to an energy generator (known and not depicted), such as a radiofrequency generator. For instance, to maintain compatibility with conventional transseptal accessory devices (known and not depicted), the outer diameter of the distal section (of the elongated medical needle 102) may be less than about 0.032 inches. The inner diameter of the open lumen 104 may be at least about 0.014 inches.

The lid assembly 108 is configured to (at least partially) occlude the open lumen 104 (which may or may not be electrically conductive for emitting energy). Tissue coring may be mitigated (reduced, preferably non-existent) as the active surface (the energy emitting surface) of the lid assembly 108 contacts (at least in part) the biological feature (tissue).

In accordance with an option, tissue coring may be mitigated (reduced, preferably non-existent) as the active surface (the energy emitting surface) of the lid assembly 108 contacts (at least in part) the biological feature (tissue) while the distal face 106 may be partially open.

Referring to the embodiment (implementation) as depicted in FIG. 5, the lid assembly 108 is configured to be received, and stored, in a storage compartment 112 (that is, in a storage position located within a storage compartment 112). The storage compartment 112 is defined, preferably, in and along a side wall 105 of the open lumen 104. The storage compartment 112 extends along a length of the side wall 105. As depicted in FIG. 5, the lid assembly 108 has been removed from (at least in part), or deployed from, the storage compartment 112 (that is, removed from the storage position); this is done (preferably) in such a way that the lid assembly 108 is deployed (and positioned), at least in part, flush against the distal face 106 of the open lumen 104 (in a lid-closed position). The lid assembly 108 includes, preferably, the shaped wire structure 110 having an electrically conductive material (once or after the lid assembly 108 has been deployed (removed) from the storage compartment 112 and the lid assembly 108 is placed flush against the distal face 106 of the open lumen 104. It will be appreciated that while the lid assembly 108 is deployed from the storage compartment 112 to the deployment position, the lid assembly 108 remains selectively inactivated and does not emit energy (such as radiofrequency energy). The shaped wire structure 110 (also called an exposed wire structure) includes, for instance, a wire coil or wire spiral configuration.

Referring to the embodiment (implementation) as depicted in FIG. 6, the lid assembly 108 is selectively activated to emit energy (such as radiofrequency energy) after the lid assembly 108 is deployed and in the deployment position. It will be appreciated that the shaped wire structure 110 has a thickness sufficient for the purposes of emitting energy, and preferably maximizing tissue vaporization of the biological feature 900 (such as the septum) without, preferably, forming a tissue core, or forming a tissue core of insignificant size that does little if any harm to the patient.

Referring to the embodiment (implementation) as depicted in FIG. 6, the lid assembly 108 includes a wire with a coiled distal end. The lid assembly 108 is configured to be selectively movable from the covering position (as depicted in FIG. 4) to an exposing position (as depicted in FIG. 5 or FIG. 6) in which the lid assembly 108 exposes, at least in part, the distal face 106 of the open lumen 104; this is done in such a way that the lid assembly 108, in use, permits selective fluid communication between the open lumen 104 and the biological feature 900 after the lid assembly 108, in use, selectively exposes, at least in part, the distal face 106 of the open lumen 104. An advantage of this arrangement is that it allows, at least in part, the injection of a contrast material to the open lumen 104, and/or the detection of a pressure reading (if so desired).

Referring to the embodiment (implementation) as depicted in FIG. 6, the lid assembly 108 includes the shaped wire structure 110 with a coiled distal end. The shaped wire structure 110 presents a coil-shaped active surface configured to vaporize a circular area of the biological feature 900.

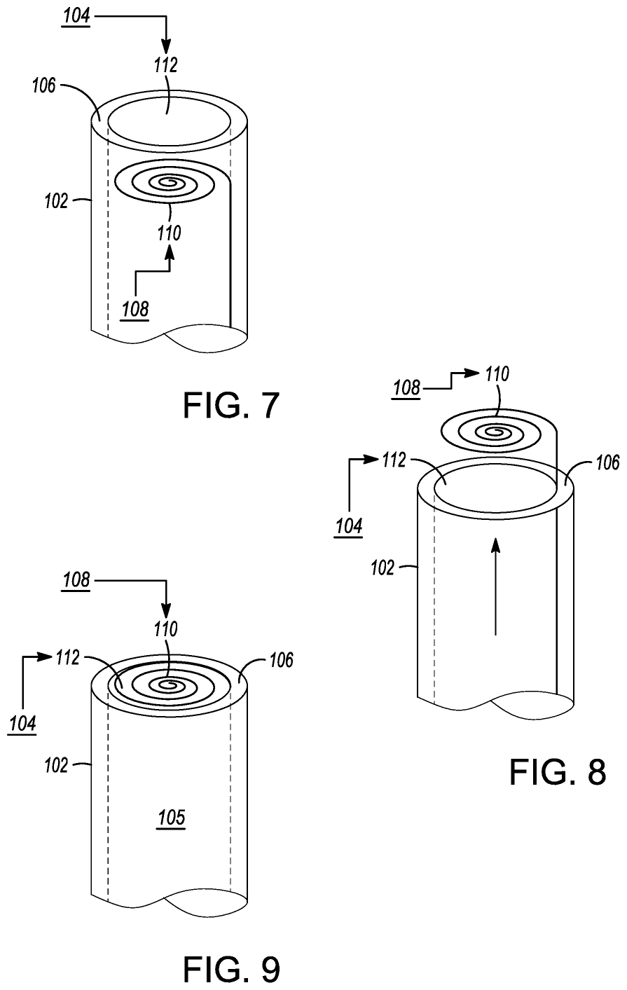

FIG. 7, FIG. 8 and FIG. 9 depict perspective views of embodiments (implementations) of the elongated medical needle 102 of FIG. 4 (an embodiment of a workflow associated with the elongated medical needle 102 is depicted). It will be appreciated that the embodiments associated with FIG. 5 and FIG. 6 are similar to the embodiments associated with FIG. 7, FIG. 8 and FIG. 9.

Referring to the embodiment (implementation) as depicted in FIG. 7, the lid assembly 108 (depicted as the shaped wire structure 110) is positioned within an interior wall of the open lumen 104. It will be appreciated that for FIG. 7, FIG. 8 and FIG. 9, the shaped wire structure 110 is formed as a coil structure or a pre-set coil structure. The shaped wire structure 110 is formed as a pre-set arrangement (that is, whether the shaped wire structure 110 is positioned in a storage position, as depicted in FIG. 7, or positioned in a deployed position as depicted in FIG. 8 or FIG. 9). The straight portion (that extends down the elongated medical needle 102) is housed in an internal lumen of the elongated medical needle 102 either by a sleeve or some other form of housing, etc. For the embodiments of FIG. 7, FIG. 8 and FIG. 9, the lid assembly 108 (or the shaped wire structure 110) is extended away from the distal portion of the elongated medical needle 102 so that the pre-set coil structure is positioned at (or proximate to) the distal face 106 (or retracted back into the interior of the elongated medical needle 102, etc.).

Referring to the embodiment (implementation) as depicted in FIG. 8, the lid assembly 108 (depicted as the shaped wire structure 110) is advanced along the open lumen 104 toward the distal face 106.

Referring to the embodiment (implementation) as depicted in FIG. 9, the lid assembly 108 has been removed from the deployment position (the storage compartment 112 is the interior of the open lumen 104). The lid assembly 108 is deployed (and positioned), at least in part, flush against the distal face 106 of the open lumen 104 (in a lid-closed position). The lid assembly 108 may be retracted slightly to cover the distal face 106 of the open lumen 104 until a lid-closed position is (substantially) achieved. The lid assembly 108 (depicted as the shaped wire structure 110) retains (or forms) a coiled arrangement (geometry) through shape setting, leveraging shape-memory alloys such as Nitinol or cold worked stainless steel.

FIG. 10, FIG. 11 and FIG. 12 depict schematic views of embodiments (implementations) of the elongated medical needle 102 of FIG. 4.

Referring to the embodiment (implementation) as depicted in FIG. 10 to FIG. 12, the elongated medical needle 102 is configured to be inserted into a confined space or a tortuous space defined by the living body (the patient). The elongated medical needle 102 is (preferably) impermeable by a bodily fluid located in the confined space defined by the living body.

Referring to the embodiment (implementation) as depicted in FIG. 10, the needle/wire system is advanced up the Inferior Vena Cava (IVC) into the right ventricle.

Referring to the embodiment (implementation) as depicted in FIG. 11, a physician may use an existing method (called the two-drop method) to locate the fossa ovalis on the septum.

Referring to the embodiment (implementation) as depicted in FIG. 12, radiofrequency energy is applied to puncture the septum wall. The lid assembly 108 is utilized to puncture the tissue and/or may mitigate tissue coring. The lid assembly 108 is retracted (back into the storage position) once the lid assembly 108 is no longer needed and is deactivated. Preferably, the lid assembly 108 (or the coil formation) may be used as a pigtail anchor once the lid assembly 108 has crossed the fossa of the heart. The elongated medical needle 102 may be configured to act as a guidewire that may be anchored in the left side of the heart. The next step may be to release the lid assembly 108 from the open lumen needle, then use the guidewire to install and/or deploy at least one accessory device for further treatment (if required). The workflow of the elongated medical needle 102 may be similar to the VERSACROSS (TRADEMARK) RF Transseptal Device manufactured by the BAYLIS (TRADEMARK) Company (headquartered in Canada).

The elongated medical needle 102 may temporarily maintain puncture location until the position might be anchored using an accessory guidewire (known and not depicted).

Referring to the embodiment (implementation) as depicted in FIG. 12, the medical needle 102 is configured for (preferably) supporting cardiac procedures, allowing the user to perform transseptal puncture across the fossa ovalis in the heart (of the patient).

Referring to the embodiments (implementations) as depicted in FIG. 10 to FIG. 12, there is depicted a method. The method is for emitting energy (such as radiofrequency energy) toward the biological feature 900 of the patient 902. As depicted in FIG. 10, the method includes and is not limited to (comprises) maneuvering and positioning a distal face 106 of an open lumen 104 of a medical needle 102 toward and proximate to the biological feature 900 of the patient 902. As depicted in FIG. 11, the method also includes selectively covering, at least in part, the distal face 106 of the open lumen 104 with a lid assembly 108. As depicted in FIG. 11 and FIG. 12, the method also includes selectively emitting energy toward the biological feature 900 from the lid assembly 108 (this is done, preferably after the distal face 106 has been maneuvered toward, and positioned proximate to, the biological feature 900 and after the lid assembly 108, in use, selectively covers, at least in part, the distal face 106 of the open lumen 104); this is done, preferably, in such a way that the lid assembly 108, in use, vaporizes a portion of the biological feature 900.

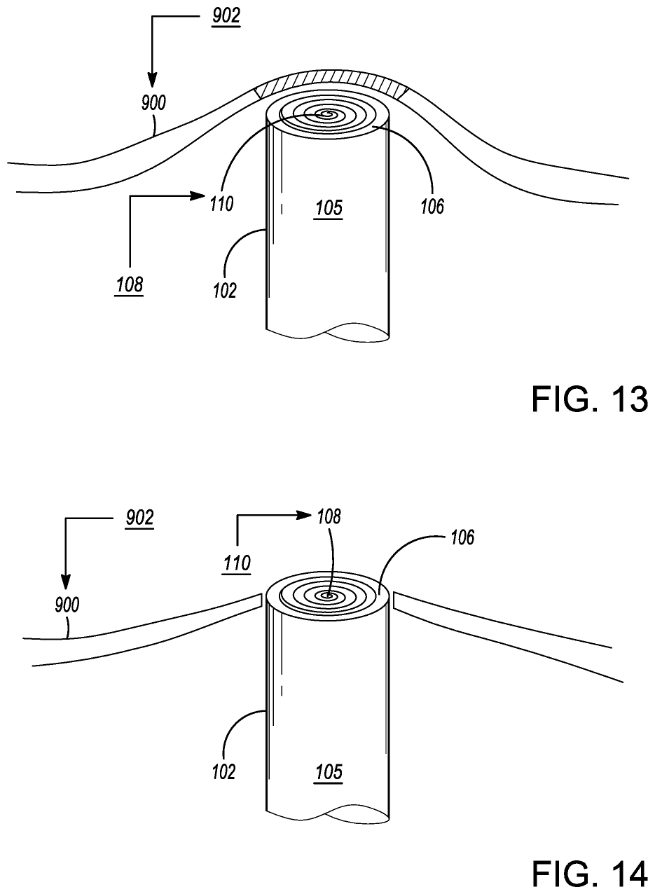

FIG. 13 and FIG. 14 depict perspective views of embodiments (implementations) the medical needle 102 of FIG. 4.

Referring to the embodiment (implementation) as depicted in FIG. 13, the distal portion of the elongated medical needle 102 is positioned to tent the fossa ovalis.

The lid assembly 108 vaporizes an area of the tissue, preferably without introducing any free-floating particulates of tissue (in sharp contrast to FIG. 2).

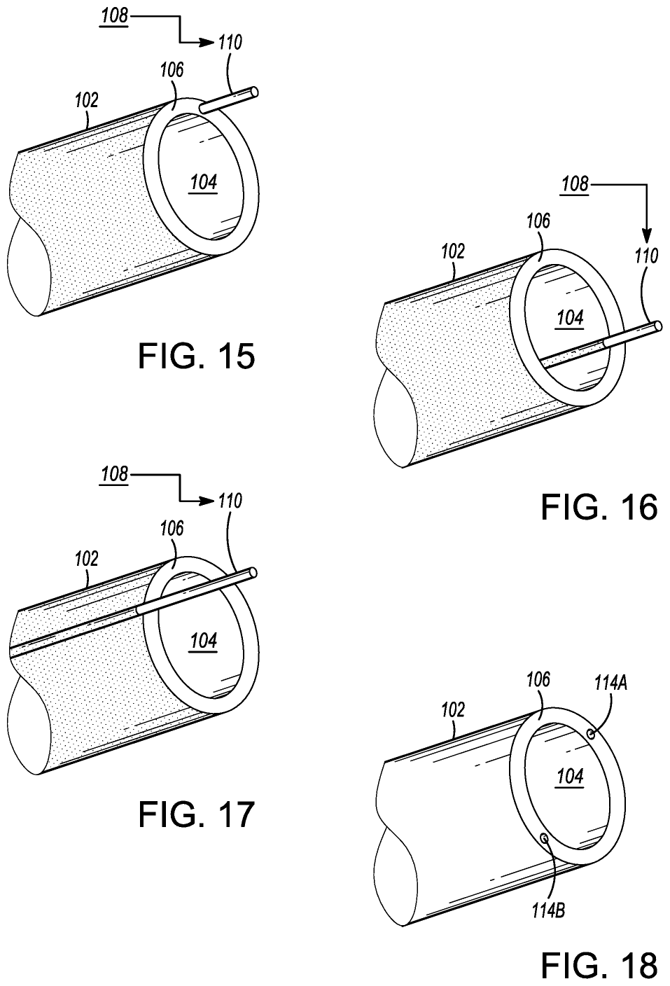

FIG. 15, FIG. 16, FIG. 17 and FIG. 18 depict perspective views of embodiments (implementations) the medical needle 102 of FIG. 4.

Referring to the embodiments (implementations) as depicted in FIG. 15 to FIG. 18, there are depicted embodiments for housing the lid assembly 108 within or against the interior and/or the exterior of the elongated medical needle 102.

Referring to the embodiment (implementation) as depicted in FIG. 15, the lid assembly 108 (or the shaped wire structure 110) is housed within the wall (the side wall) of the elongated medical needle 102. It will be appreciated that the shaped wire structure 110 may have an electrical insulation layer (at least in part or in whole) if desired or may be without an electrical insulation layer (at least in part or in whole) if desired.

In accordance with an option, another lumen may be positioned within the open lumen 104 for possible prevention of wall fracture, and/or mitigates against buckling of the lid assembly 108 as it is advanced/retracted through the elongated medical needle 102.

Referring to the embodiment (implementation) as depicted in FIG. 16, the lid assembly 108 and/or the shaped wire structure 110 is/are positioned flush against the inside of the open lumen 104 of the elongated medical needle 102.

A layer of heatshrink may be positioned to electrically insulate the inside surface of the open lumen 104. In accordance with an option, a layer of heatshrink (or other suitable coating) may be applied to adhere the shaped wire structure 110 and/or to the inside surface of the open lumen 104 (if so desired).

Referring to the embodiment (implementation) as depicted in FIG. 17, the lid assembly 108 and/or the shaped wire structure 110 is/are fixed to the outside surface of the elongated medical needle 102.

Referring to the embodiment (implementation) as depicted in FIG. 18, there is depicted an option for the embodiment as depicted in FIG. 15. The lumens (114A, 114B) are formed in the wall of the elongated medical needle 102, and are configured to deliver liquid and/or deploy a guidewire (known and not depicted).

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. Apparatus for use with a biological feature of a patient, the apparatus comprising:
an elongated medical needle defining an open lumen presenting a distal face configured to be maneuvered toward, and positionable proximate to, the biological feature of the patient; and
a lid assembly configured to selectively cover, at least in part, the distal face of the open lumen; and
the lid assembly also configured to puncture through the biological feature; and
the lid assembly also configured to selectively uncover, at least in part, the distal face of the open lumen after the lid assembly has punctured through the biological feature.

2. The apparatus of claim 1, wherein:
the elongated medical needle is configured to house the lid assembly.

3. The apparatus of claim 1, wherein:
the lid assembly includes a mechanical puncturing portion.

4. The apparatus of claim 1, wherein:
the lid assembly includes an active surface.

5. The apparatus of claim 1, wherein:
the lid assembly is configured to be selectively movable from a covering position to an exposing position in which the lid assembly exposes, at least in part, the distal face of the open lumen in such a way that the lid assembly, in use, permits selective fluid communication between the open lumen and the biological feature after the lid assembly, in use, selectively exposes, at least in part, the distal face of the open lumen.

6. The apparatus of claim 1, wherein:
the lid assembly is operatively mounted at the distal face of the open lumen of the elongated medical needle.

7. An apparatus for use with a biological feature of a patient, the apparatus comprising:
an elongated medical needle defining an open lumen presenting a distal face configured to be maneuvered toward, and positionable proximate to, the biological feature of the patient; and
a lid assembly configured to selectively cover, at least in part, the distal face of the open lumen; and
the lid assembly also configured to selectively emit energy toward the biological feature after the distal face has been maneuvered toward, and positioned proximate to, the biological feature and after the lid assembly, in use, selectively covers, at least in part, the distal face of the open lumen in such a way that the lid assembly, in use, vaporizes a portion of the biological feature.

8. The apparatus of claim 7, wherein:
the distal face is configured to selectively emit energy.

9. The apparatus of claim 7, wherein:
the elongated medical needle is configured to house the lid assembly.

10. The apparatus of claim 7, wherein:
the lid assembly includes a wire with a coiled distal end.

11. The apparatus of claim 7, wherein:
the lid assembly is configured to be selectively movable from a covering position to an exposing position in which the lid assembly exposes, at least in part, the distal face of the open lumen in such a way that the lid assembly, in use, permits selective fluid communication between the open lumen and the biological feature after the lid assembly, in use, selectively exposes, at least in part, the distal face of the open lumen.

12. The apparatus of claim 7, wherein:
the lid assembly is operatively mounted at the distal face of the open lumen of the elongated medical needle.

13. The apparatus of claim 7, wherein:
the lid assembly includes:
a shaped wire structure with a coiled distal end; and
the shaped wire structure presents a coil-shaped active surface configured to vaporize a circular area of the biological feature.

14. The apparatus of claim 7, wherein:
the lid assembly includes a shaped wire structure; and
the elongated medical needle is configured to house a shaped wire structure.

15. A method of emitting energy toward a biological feature of a patient, the method comprising:
maneuvering and positioning a distal face of an open lumen of an elongated medical needle toward and proximate to the biological feature of the patient; and
selectively covering, at least in part, the distal face of the open lumen with a lid assembly; and
selectively emitting energy toward the biological feature from the lid assembly after the distal face has been maneuvered toward, and positioned proximate to, the biological feature and after the lid assembly, in use, selectively covers, at least in part, the distal face of the open lumen in such a way that the lid assembly, in use, vaporizes a portion of the biological feature.
